(19) United States
(12) Patent Application Publication
(10) Pub. No.: US 2014/0085607 A1
(43) Pub. Date: Mar. 27, 2014

(54) IMAGE DETECTING APPARATUS AND IMAGE DETECTING METHOD

(71) Applicant: ALTEK CORPORATION, Hsinchu City (TW)

(72) Inventors: Pin-Wen Chen, Changhua County (TW); Ching-Chung Hsu, Kaohsiung City (TW); Tso-Yu Chang, New Taipei City (TW); Chung-Ping Lai, Taichung City (TW)

(73) Assignee: ALTEK CORPORATION, Hsinchu City (TW)

(21) Appl. No.: 13/717,726

(22) Filed: Dec. 18, 2012

(30) Foreign Application Priority Data
Sep. 24, 2012 (TW) 101134918

(51) Int. Cl. A61B 3/14; A61B 3/15

(52) U.S. Cl. CPC A61B 3/14; A61B 3/156; A61B 3/152
USPC 351/207; 351/206; 351/246; 351/208

(57) ABSTRACT

An image detecting apparatus for detecting a first eyeball is provided. The image detecting apparatus includes an illumination light source, an imaging lens, an image sensing device, a display and a viewfinder. The illumination light source emits an illumination beam, and the illumination beam irradiates the first eyeball. The first eyeball reflects the illumination beam into an image beam. The imaging lens is disposed on a transmission path of the image beam. The image sensing device is disposed on the transmission path of the image beam, wherein the imaging lens is disposed between the first eyeball and the image sensing device. The display shows the image formed by the image beam. The viewfinder is disposed in front of the display such that a second eyeball observes the display via the viewfinder. An image detecting method is also provided.

### CROSS-REFERENCE TO RELATED APPLICATION

This application claims the priority benefit of Taiwan application serial no. 101134918, filed on Sep. 24, 2012. The entirety of the above-mentioned patent application is hereby incorporated by reference herein and made a part of this specification.

### BACKGROUND

1. Field of the Invention

The invention relates to an image detecting apparatus and an image detecting method, and more particularly to, an image detecting apparatus and an image detecting method for detecting an eyeball.

2. Description of Related Art

In recent years, eye related diseases, along with life-styles of people, have gradually increased. Eye diseases such as a retinal detachment caused by a high myopia or a macular degeneration caused by chronic diseases such as diabetes have influenced lives of many patients. In these common eye diseases, clinical diagnosis methods thereof are mostly observing and shooting a retina, a macula and distributions of microvessels thereof via a desktop ophthalmoscope. When a fundus appears to have an angiogenesis or a vascular rupture, such as symptoms of macular degeneration haemorrhagic, deaths of photosensitive and color discriminating cone cells and rod cells on the retina may easily occur, thus causing the patient to lose vision. Therefore, in the diagnosis of eye diseases and preventive cares, observation and tracking of images of the fundus is extremely important.

In general, due to a pupil size limit of human eye, when observing the image shooting of the fundus, a drug, such as a mydriatic agent, is administered for relaxing ciliary muscles in order to dilate the pupil; and the images of the fundus with a broader perspective for a diagnosis may still required to be captured with a plurality of angles and after multiple shootings. However, the conventional ophthalmoscope has a large volume and expensive price, and it uses visible light for illumination. Under a condition that the pupil of the patient is dilated, continuously shooting the images of the fundus with visible light may increase burden or produce discomfort on the patient eye. In addition, when shooting the images of the fundus, professional health care workers are required for operating the ophthalmoscope, and by multiple shootings that clearer and complete images of the fundus may be obtained. Difficulty in shooting the images of the fundus and time required for the shooting often make it difficult to track a state of the patient fundus, and may even more delay treatment timing, thereby causing regret. Therefore, conveniently attaining the fast, complete and clear images of the fundus is currently the urgent issues in the field of eye care medical.

### SUMMARY OF THE INVENTION

The invention provides an image detecting apparatus capable of being configured to detecting the image of an eyeball.

The invention provides an image detecting method enabling the user to self-detect the image of an eyeball thereof.

An image detecting apparatus for detecting a first eyeball is provided in an embodiment of the invention. The image detecting apparatus includes an illumination light source, an imaging lens, an image sensing device, a display and a viewfinder. The illumination light source emits an illumination beam, the illumination beam irradiates the first eyeball, and the first eyeball reflects the illumination beam into an image beam. The imaging lens is disposed on a transmission path of the image beam. The image sensing device is disposed on the transmission path of the image beam, wherein the imaging lens is disposed between the first eyeball and the image sensing device. The display shows an image formed by the image beam that are sensed by the image sensing device. The viewfinder is disposed in front of the display such that a second eyeball observes the display via the viewfinder.

In an embodiment of the invention, the illumination beam and the image beam are invisible beams.

In an embodiment of the invention, the image detecting apparatus further includes a shading piece disposed on the imaging lens and surrounding a space between the imaging lens and the first eyeball.

In an embodiment of the invention, the image detecting apparatus further includes a control unit and a user interface. The control unit is electrically connected to the image sensing device. The user interface is electrically connected to the control unit, wherein when the control unit determines that an intensity of an ambient light detected by the image sensing device is greater than a preset value, the control unit reminds the user to adjust a shading piece via the user interface, such that the shading piece is closely fit on the first eyeball.

In an embodiment of the invention, the image detecting apparatus further includes a control unit electrically connected to the imaging lens and the image sensing device, wherein the control unit commands the imaging lens to focus on a pupil of the first eyeball. When the control unit determines that an image size of the pupil sensed by the image sensing device is greater than a preset value, the control unit enters a standby shooting state.

In an embodiment of the invention, when the control unit enters the standby shooting state, the control unit commands the imaging lens to focus on a fundus of the first eyeball.

In an embodiment of the invention, the image detecting apparatus further includes a user interface, wherein when the control unit enters the standby shooting state, the control unit informs the user to start shooting via the user interface.

In an embodiment of the invention, the image detecting apparatus further includes a first body and a second body. The first body carries the illumination light source, the imaging lens and the image sensing device. The second body carries the display and the viewfinder, wherein the second body is adapted to be detachably bound to or rotatably bound to a first position or a second position on the first body. When the second body is bound to the first position on the first body, the imaging lens and the viewfinder face towards a same direction. When the second body is bound to the second position on the first body, the imaging lens and the viewfinder respectively face towards opposite directions.

In an embodiment of the invention, when the second body is bound to the first position on the first body, the imaging lens and the viewfinder respectively face towards the first eyeball and the second eyeball, and the first eyeball and the second eyeball both belong to a same user.

In an embodiment of the invention, when the second body is bound to the second position on the first body, the imaging lens and the viewfinder respectively face towards the first eyeball and the second eyeball, and the first eyeball and the second eyeball respectively belong to a subject and an operator.

In an embodiment of the invention, when the second body is bound to the second position on the first body, the display moves from within the second body to an external part of the second body, the imaging lens and the display respectively face towards the first eyeball and second eyeball, and the first eyeball and the second eyeball respectively belong to a subject and an operator.

In an embodiment of the invention, the first body has a first handle and the second body has a second handle. When the second body is bound to the first position on the first body, the first handle and the second handle are respectively located at two opposite sides of the image detecting apparatus, so as to be respectively gripped by two hands of a user.

In an embodiment of the invention, at least one of the first body and the second body has a foot stand fixing hole for fixing a foot stand configured to support the image detecting apparatus.

In an embodiment of the invention, the image detecting apparatus further includes a control unit electrically connected to the image sensing device and the display. When the control unit determines that the pupil of the first eyeball sensed by the image sensing device is deviated from a central region of the image sensing device, the control unit commands the display to show a prompting sign to inform the user on how to move a relative position of the imaging lens with respect to the first eyeball.

In an embodiment of the invention, an image detecting method is provided. The image detecting method includes providing an illumination beam to a first eyeball, wherein the first eyeball reflects the illumination beam into an image beam. The image detecting method also includes detecting an image carried by the image beam. The image detecting method further includes showing the image carried by the image beam to a second eyeball, wherein the first eyeball and the second eyeball belong to a same user.

In an embodiment of the invention, the image detecting method further includes, before detecting the image carried by the image beam, determining an intensity of an ambient light reflected by the first eyeball. When the intensity of the ambient light is greater than a preset value, the user is reminded to adjust a relative position of a shading piece with respect to the first eyeball until the intensity of the ambient light is less than the preset value.

In an embodiment of the invention, the image detecting method further includes determining whether a pupil of the first eyeball is greater than a preset value or not according to the image carried by the image beam, wherein if not, then the user is reminded to adjust the relative position of a shading piece with respect to the first eyeball.

In an embodiment of the invention, if the pupil of the first eyeball is greater than the preset value, then a fundus of the first eyeball is detected.

In an embodiment of the invention, if the pupil of the first eyeball is greater than the preset value, then the user is informed to start shooting a fundus of the first eyeball.

In an embodiment of the invention, the image detecting method further includes providing the illumination beam to the second eyeball, wherein the second eyeball reflects the illumination beam into another image beam. The image detecting method also includes detecting an image carried by the another image beam. The image detecting method also includes showing the image carried by the another image beam to the first eyeball.

According to the foregoing, the image detecting apparatus in the embodiments of the invention may use the illumination light source to illuminate one of the eyes of the user, transmit the eye image to the sensing device to form an image thereon via the imaging lens, and to show the eye image sensed by the image sensing device on the display, so as to be observed by the other eye of the user or an eye of another operator, thereby assisting the user to shoot the eye image by oneself or by others. The image detecting method in the embodiments of the invention may enable the user to detect the image of one of the eyes, and may use the other eye of user to ensure the detected eye image, so as to assist the user to adjust a shooting range.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of an image detecting apparatus according to an embodiment of the invention.

FIG. 2A to FIG. 2C are schematic diagrams of a control unit in the embodiment of FIG. 1, which uses a display to show corresponding prompting signs according to imaging locations of a pupil on the image sensing device.

FIG. 3A is a schematic diagram of an image detecting apparatus according to another embodiment of the invention.

FIG. 3B is a schematic diagram illustrating a jointing-separating method of the image detecting apparatus according to the embodiment in FIG. 3A.

FIG. 3C is a schematic diagram illustrating a variation of the image detecting apparatus of FIG. 3A.

FIG. 4 is an operation schematic diagram of the image detecting apparatus of FIG. 3C.

FIG. 5A is a schematic diagram illustrating a variation of the image detecting apparatus of FIG. 4.

FIG. 5B is a schematic diagram lustrating a variation of the image detecting apparatus of FIG. 3A.

FIG. 6A and FIG. 6B are schematic diagrams illustrating variations of the image detecting apparatus of FIG. 3A.

FIG. 7 is a flow chart diagram of an image detecting method according to an embodiment of the invention.

FIG. 8 is a flow chart diagram illustrating step variations in the image detecting method of FIG. 7.

### DETAILED DESCRIPTION OF DISCLOSED EMBODIMENTS

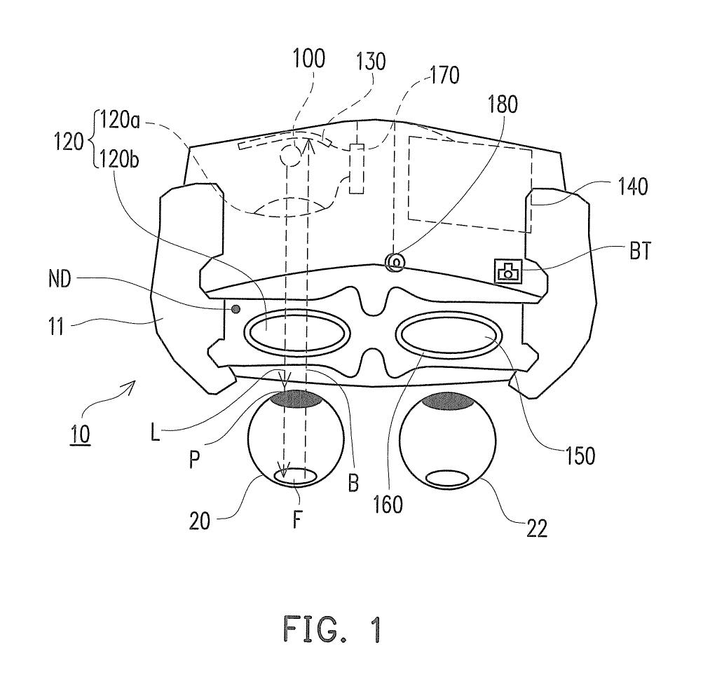

FIG. 1 is a schematic diagram of an image detecting apparatus according to an embodiment of the invention. Referring to FIG. 1, in this embodiment, an image detecting apparatus 10 for detecting a first eyeball 20 is provided. The image detecting apparatus 10 includes an illumination light source 100, an imaging lens 120, an image sensing device 130, a display 140 and a viewfinder 150. In this embodiment, the image detecting apparatus 10 may include a casing 11, wherein the illumination light source 100, the imaging lens 120, the image sensing device 130, the display 140 and the viewfinder 150 may be disposed within the casing 11. The illumination light source 100 emits an illumination beam L, the illumination beam L irradiates the first eyeball 20, and the first eyeball 20 reflects the illumination beam L into an image beam B. For example, the illumination beam L irradiates a fundus F of the first eyeball 20, and the fundus F of the first eyeball 20 reflects the illumination beam L into the image beam B. In this embodiment, the illumination light source 100 is disposed within the casing 11, and the illumination beam L irradiates the first eyeball 20 via the imaging lens 120. However, in other embodiments, the illumination light source 100 may also be disposed on the casing 11 or other suitable positions within the casing 11 in order to illuminate the fundus F of the first eyeball 20, and the invention is not limited thereto. The imaging lens 120 is disposed on a transmission path of the image beam B, wherein the imaging lens 120 may have one or more lenses; and in this embodiment, two are taken as an example; namely, the imaging lens 120 may include a lens 120a and a lens 120b, but the invention is not limited thereto. The image sensing device 130 is disposed on the transmission path of the image beam B, wherein the imaging lens 120 is disposed between the first eyeball 20 of the user and the image sensing device 130. The display 140 shows the image formed by the image beam B that are sensed by the image sensing device 130, and the display 140 may be a liquid crystal display (LCD). However, in other embodiments, the display 140 may also be an organic light-emitting diode display (OLED display) or other suitable displays. The viewfinder 150 is disposed in front of the display 140 such that a second eyeball 22 may observe the display 140 via the viewfinder 150. In this embodiment, the illumination beam L and the image beam B may be invisible beams, such as infrared lights; however, in other embodiments, the illumination beam L and the image beam B may also be visible lights, and the invention is not limited thereto. Moreover, the image detecting apparatus 10 may further include a shading piece 160 disposed on the imaging lens 120 and surrounding a space between the imaging lens 120 and the first eyeball 20. In addition, the shading piece 160 may also be disposed outside of the viewfinder 150. For example, the shading piece 160 may be produced from an opaque material such as silicone or rubber, the eye of the user may lie against thereon, and may shade off the light from the outside so that a dark environment is formed between the first eyeball 20 of the user and the image detecting apparatus 10, and thus may reduce an influence of an ambient light to the first eyeball 20. When the first eyeball 20 of the user is under the dark environment, a pupil P of the first eyeball 20 may naturally be dilated, such that the fundus F of the first eyeball 20 may be well irradiated by the illumination beam L, and thus the image may be obtained. In this embodiment, since the illumination beam L and the image beam B may be the invisible beams, the first eyeball 20 of the user does not easily constrict the pupil P as react to the illumination beam L and the image beam B, and with the shading piece 160 shielding the ambient light, the pupil of the user may still have an adequate size to shoot the image of the fundus with a wide viewing angle under a condition of not administering a drug, such as a mydriatic agent. In this way, side effects brought by using the mydriatic agent may be avoided, and burdens on the user may be reduced.

In detail, in this embodiment, the image detecting apparatus 10 further includes a control unit 170 and a user interface 180. The control unit 170 is electrically connected to the image sensing device 130. The user interface 180 is electrically connected to the control unit 170. When the control unit 170 determines that an intensity of an ambient light detected by the image sensing device 130 is greater than a preset value, the control unit 170 reminds the user to adjust a shading piece 160 via the user interface 180, such that the shading piece 160 is closely fit on the first eyeball 20. For example, the user interface 180 may be a display, such as a light emitting diode (LED) display or an organic light emitting diode (OLED) display. When the eye of the user is not in a favorable contact with the shading piece 160, thereby causing the ambient light to enter the image detecting apparatus 10 and to be detected by the image sensing device 130, and when the control unit 170 determines that the ambient light received by the image sensing device 130 is more than a certain light intensity, the control unit 170 may control the user interface 180 to generate a flashing bright spot in order to prompt the user to contact the eye with the shading piece 160 favorably until the control unit 170 determines that the ambient light received by the image sensing device 130 is lower than the certain light intensity. The control unit 170 may then control the user interface 180 to cancel the flashing bright spot. With this, the user may shoot the image of the fundus F of the first eyeball 20 thereof favorably under a condition without other assistance. However, in other embodiments, the user interface 180 may also be the display 140 or the flashing bright spot or image on the display 140, so that an effect similar to that of the above may also be achieved. Or, the user interface 180 may be a voice prompt system capable of prompting the user whether the focusing is achieved or it is the timing for shooting a photo of the eye. In addition, the image detecting apparatus 10 may further has a shooting button BT, such that the user may push the shooting button BT to shoot the eye image after the position of the eye is confirmed and the focusing is achieved. Moreover, in this embodiment, since the shape of the casing 11 is bilaterally symmetrical, an indicator light ND, which may include a light emitting device such as the light emitting diode or other light emitting elements, may also be disposed on the casing 11, and may be configured to indicate the user of which viewfinder may align with the first eyeball 20 (e.g., measuring eye). For example, as illustrated in FIG. 1, the indicator light ND is disposed at the top left of the viewfinder 150, which is located at the left. Thereby, a user confusion may be avoided.

In addition, the control unit 170 may also be electrically connected to the imaging lens 120 and the image sensing device 130. Wherein, the control unit 170 may command the imaging lens 120 to focus on the pupil P of the first eyeball 20. When the control unit 170 determines that an image size of the pupil P sensed by the image sensing device 130 is greater than a preset value, the control unit 170 enters a standby shooting state. Moreover, when the control unit 170 enters the standby shooting state, the control unit 170 also commands the imaging lens 120 to focus on the fundus F of the first eyeball 20. With this, the image detecting apparatus 10 may confirm whether the size of the pupil P of the first eyeball 20 of the user is sufficient for shooting the images of the fundus F in need. Furthermore, when the control unit 170 enters the standby shooting state, the control unit 170 informs the user to start shooting via the user interface 180. As a result, the user may use the image detecting apparatus 10 to auto-detect the size of the pupil P of the user oneself, and to automatically assist in prompting the user to adjust to a favorable image shooting condition. The user is enabled to solely complete the shooting of the eye images, thus increasing a convenience of shooting the eye images while still maintaining a favorable shooting quality. For example, a patient may timing detect the images of the fundus thereof at home, and in collocation of using a remote medical facility system to transfer the images of the fundus to physicians for diagnosis, and thus a follow-up observation of eye diseases may become easy.

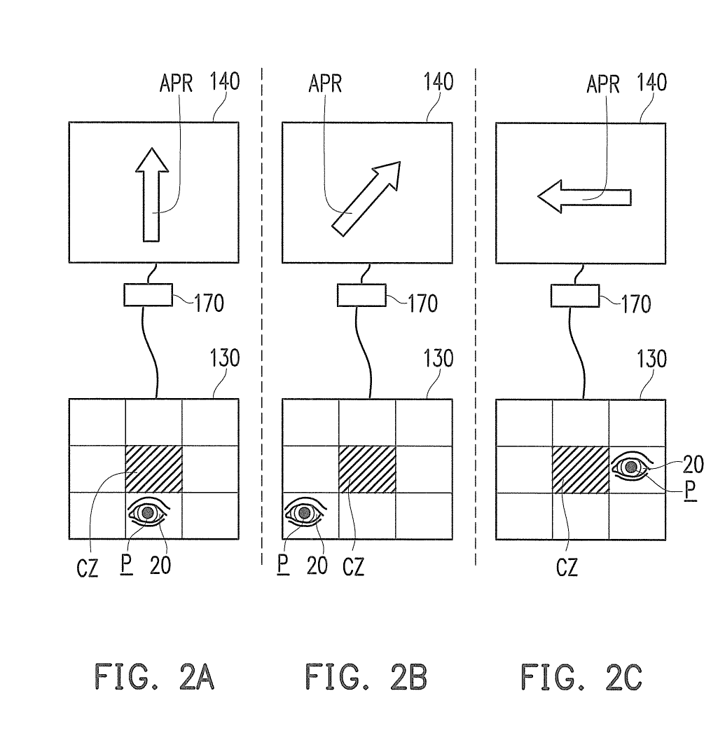

Otherwise, the control unit 170 may be electrically connected to the image sensing device 130 and the display 140. When the control unit 170 determines that the pupil P of the first eyeball 20 detected by the image sensing device 130 is deviated from a central region CZ of the image sensing device 130, the control unit 170 commands the display 140 to show a prompting sign, so as to inform the user on how to move the relative position of the imaging lens 120 with respect to the first eyeball 20. For example, referring to FIG. 2A to FIG. 2C. In FIG. 2A, when the control unit 170 determines that the imaging of the pupil P of the first eyeball 20 is located below the central region CZ of the image sensing device 130, the control unit 170 commands the display 140 to show an upward prompting sign, namely an upward arrow ARR in FIG. 2A, so as to indicate the user to adjust the relative position of the pupil P of the first eyeball 20 with respect to the imaging lens 120 upward. Similarly, as illustrated in FIG. 2B, the display 140 show an up-rightward arrow ARR capable of indicating the user to adjust the relative position of the pupil P of the first eyeball 20 with respect to the imaging lens 120 up-rightward, so that the image of the first eyeball 20 may be aligned and shot favorably. Effects illustrated in FIG. 2C are similar to the effects described above for FIG. 2A and FIG. 2B, and therefore are not repeated herein. In other embodiments, a shape and size of the central region CZ and shapes and sizes of the prompting signs showed by the display 140 may be different according to actual designs, and the invention is not limited thereto.

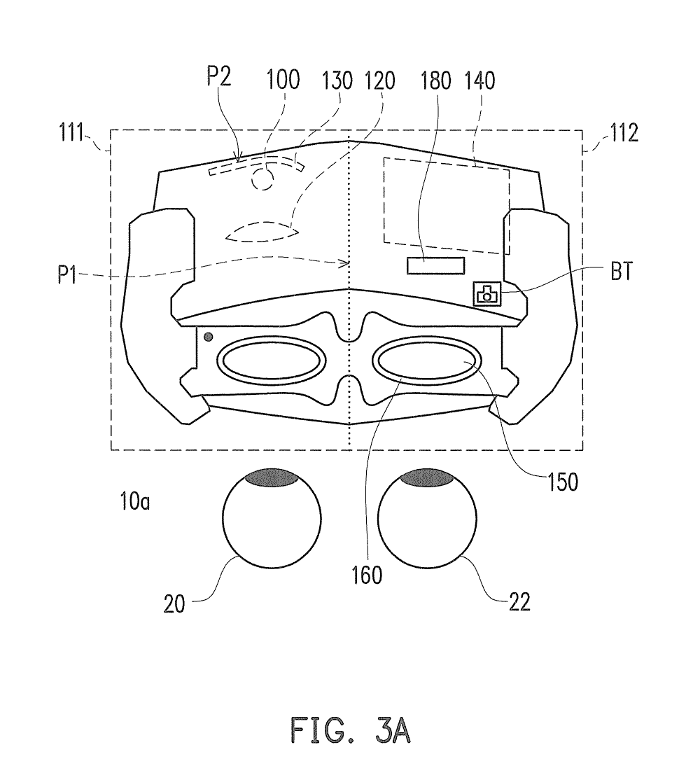

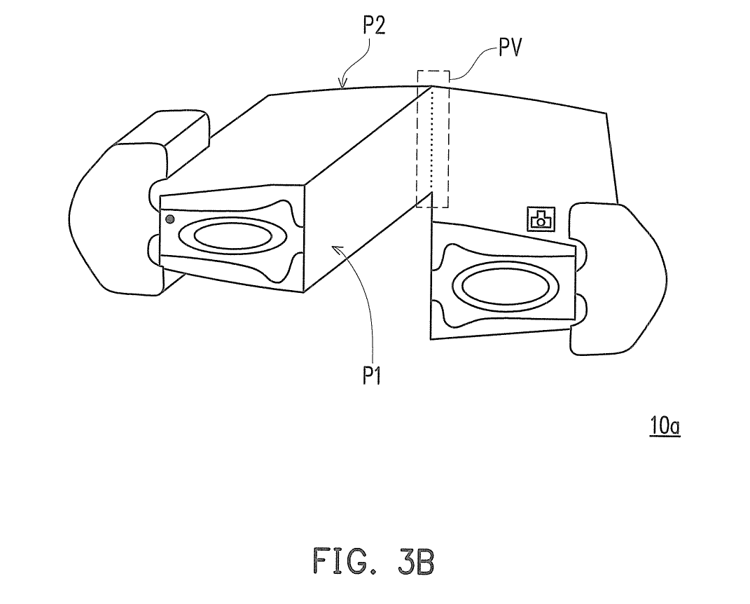

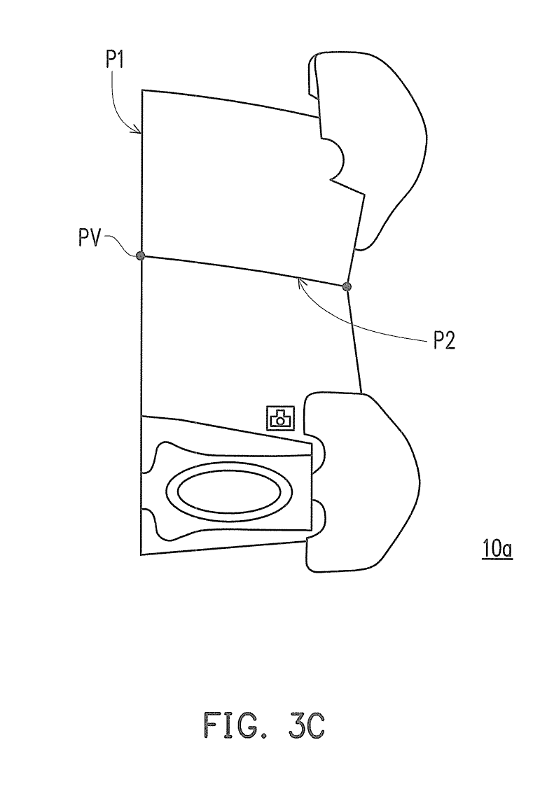

FIG. 3A is a schematic diagram of an image detecting apparatus according to another embodiment of the invention. Referring to FIG. 3A, in this embodiment, an image detecting apparatus 10a is similar to the image detecting apparatus 10 in the embodiment illustrated in FIG. 1. However, the image detecting apparatus 10a may further include a first body 111 and a second body 112. The first body 111 carries the illumination light source 100, the imaging lens 120 and the image sensing device 130. The second body 112 carries the display 140 and the viewfinder 150, wherein the second body 112 is adapted to detachably bind to a first position P1 or a second position P2 on the first body 111. Referring to FIG. 3B and FIG. 3C again. For example, in this embodiment, the first body 111 and the second body 112 may be pivotally connected via a pivot mechanism PV as illustrated in FIG. 3B, and therefore, the first body 111 may rotate in relative to the second body 112 so as to switch a binding position of the first body 111 and the second body 112, namely, the first position P1 or the second position P2. When the second body 112 is bound to the first position P1 on the first body 111, namely a condition illustrated in FIG. 3A, the imaging lens 120 and the viewfinder 150 face towards a same direction. When the second body 112 of the image detecting apparatus 10a is bound to the second position P2 on the first body 111, namely as illustrated in FIG. 3C, the imaging lens 120 and the viewfinder 150 respectively face towards opposite directions. The left and right positions, jointing and separating mechanisms and motions of the first body 111 and the second body 112 in the image detecting apparatus 10a are taken as examples for describing the embodiments of the invention, but the invention is not limited thereto.

For example, as illustrated in FIG. 3A, when the second body 112 is bound to the first position P1 on the first body 111, the imaging lens 120 and the viewfinder 150 respectively face towards the first eyeball 20 and the second eyeball 22, and the first eyeball 20 and the second eyeball 22 both belong to a same user. Namely, the user may observe the first eyeball 20 by using the firs body 111 that has the imaging lens 120 via a method similar to using binoculars, and use the second eyeball 22 to observe a prompt message showed by the user interface 180 in the second body 112, or the user may listen to a voice instruction of the user interface 180 for performing the adjustment. An example of the first eyeball 20 illustrated in FIG. 3A is a left eye of the user, and an example of the second eyeball 22 illustrated in FIG. 3A is a right eye of the use; however, in other embodiments, the first eyeball 20 may also be the right eye and the second eyeball 22 may also be the left eye, and the invention is not limited thereto. In FIG. 3A to FIG. 3C, the left eye of the user being a measured eye and the right eye of the user being an observing eye are an example for describing this embodiment, the invention is not limited thereto; and the user may also reverse the left and right of the image detecting apparatus 10a to enable the right eye to be the measured eye and the left eye to be the observing eye, and thus images of the both eyes may be measured by the user oneself.

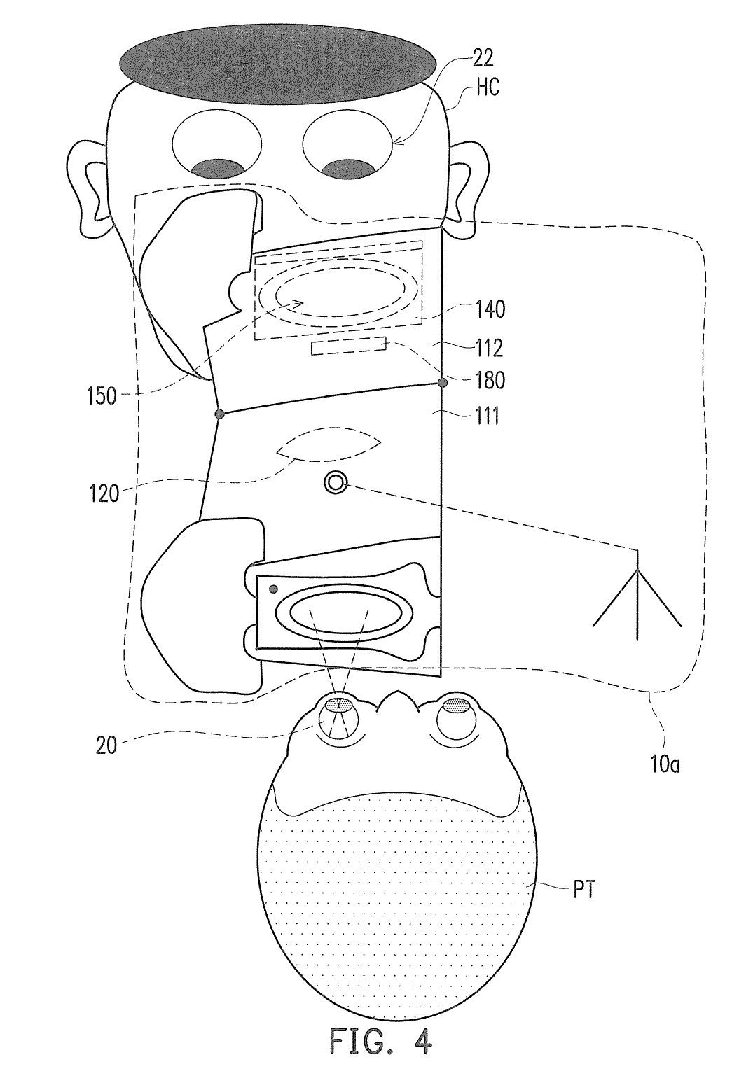

Referring to FIG. 4, when the second body 112 is bound to the second position P2 on the first body 111, the imaging lens 120 and the viewfinder 150 respectively face towards the first eyeball 20 and the second eyeball 22, and the first eyeball 20 and the second eyeball 22 respectively belong to a subject PT and an operator HC. Namely, the first eyeball 20 of the subject PT may be aligned to the first body 111 having the imaging lens 120, and the second eyeball 22 of the operator HC may be aligned to the user interface 180 in the viewfinder 150 of the second body 112 for observing the prompt message or listening to the voice instruction of the user interface 180, or may observe image of the first eyeball 20 of the subject PT from the display 140. With this, the image detecting apparatus 10a uses different binding methods of the first body 111 and the second body 112 to achieve the effect such that the user UR is enabled to shoot the images of his/her own eye, as illustrated in FIG. 3A, and to also enable the operator HC (e.g., health care personnel) to observe and shoot the image of the eye of the subject PT, as illustrated in FIG. 4. With this, a convenience in the detection of the eye images may be enhanced, and thus a quality of healthcare is improved.

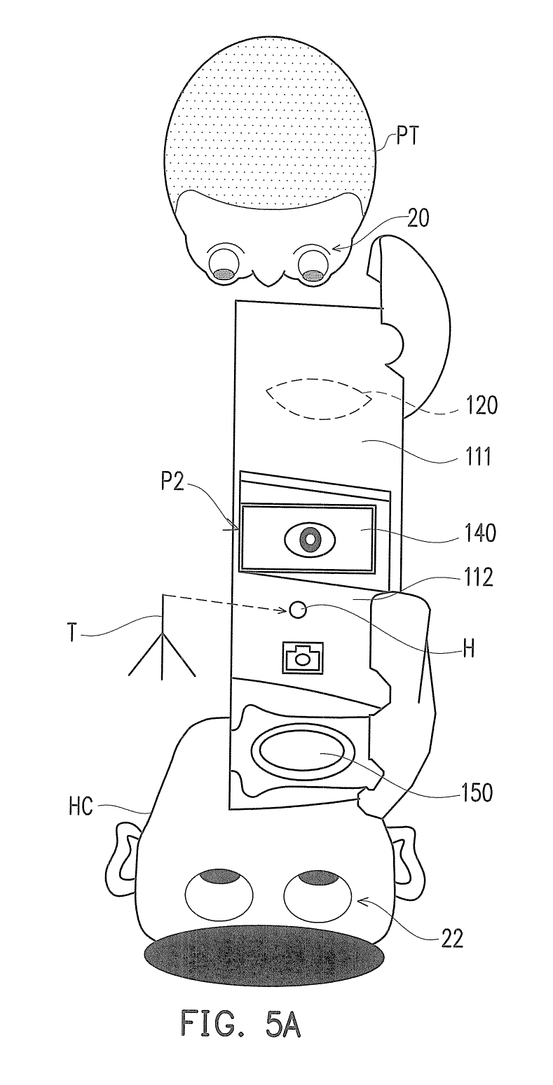

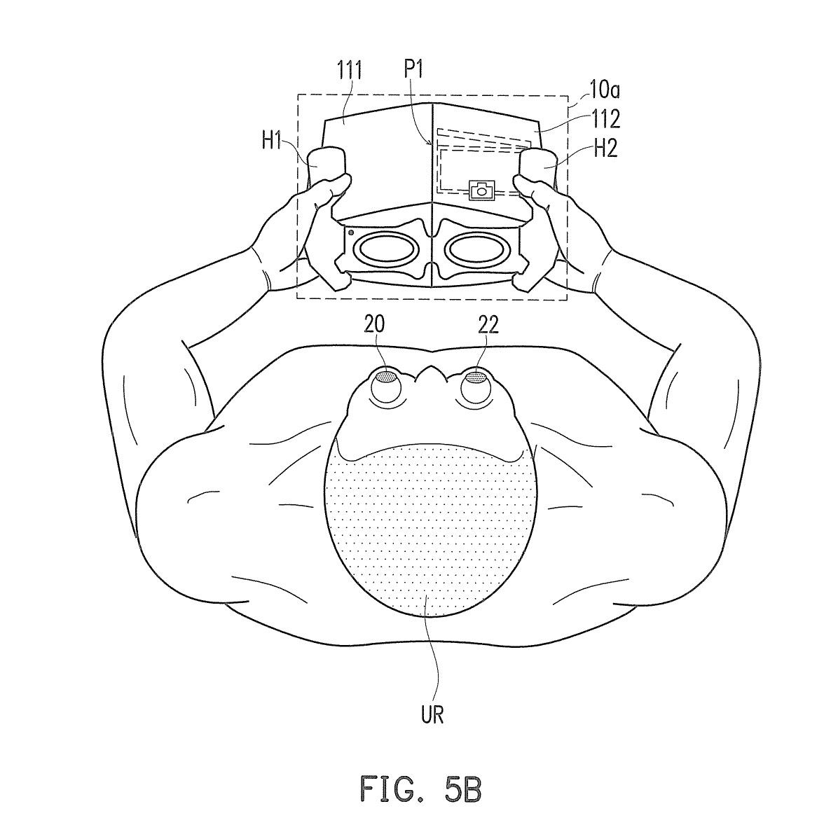

In detail, as illustrated in FIG. 5A, when the second body 112 is bound to the second position P2 on the first body 111, the display 140 may move from the second body 112 to an external part of the second body 112, the imaging lens 120 and the display 140 respectively face towards the first eyeball 20 and the second eyeball 22, and the first eyeball 20 and the second eyeball 22 respectively belong to a subject PT and an operator HC. Therefore, the operator HC may observe the display 140 located at the external part of the second body 112, and may not require to align the eye to the viewfinder 150, and thus increase the convenience in use and may avoid infections and save the costs for replacing or disinfecting the shading piece 160. In addition, at least one of the first body 111 and the second body 112 has a foot stand fixing hole H for fixing a foot stand T configured to support he image detecting apparatus. By installing the foot stand T during the shooting, the operator HC may stably detect the eye image of the subject PT and reduce situations leading to a shooting failure due to shaking. In the embodiment of FIG. 5A, the foot stand T, for example, is fixed under the first body 111, but the invention is not limited thereto.

In addition, as illustrated in FIG. 5B, the first body 111 has a first handle H1, and the second body 112 has a second handle H2. When the second body 112 is bound to the first position P1 on the first body 111, the first handle H1 and the second handle H2 are respectively located at two opposite sides of the image detecting apparatus 10a, so as to be respectively gripped by both hands of the user UR. With the first handle H1 and the second handle H2, the user UR may firmly hold the image detecting apparatus 10a favorably with both hands, and thus may increase a stability and an image quality of the eye image shot by the user oneself. Moreover, supplemented by the foot stand T, the image detecting apparatus 10a and shooting conditions of the user UR or a participant PT may be more favorable and without too much vibrations and shakings, so as to enhance a success rate and a stability for shooting the images of the fundus.

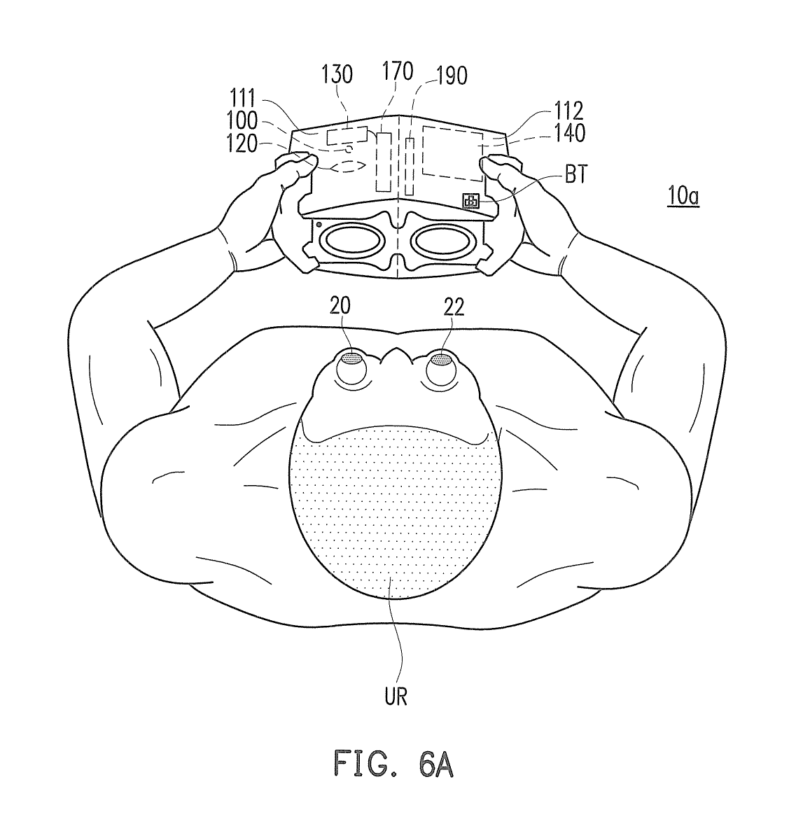

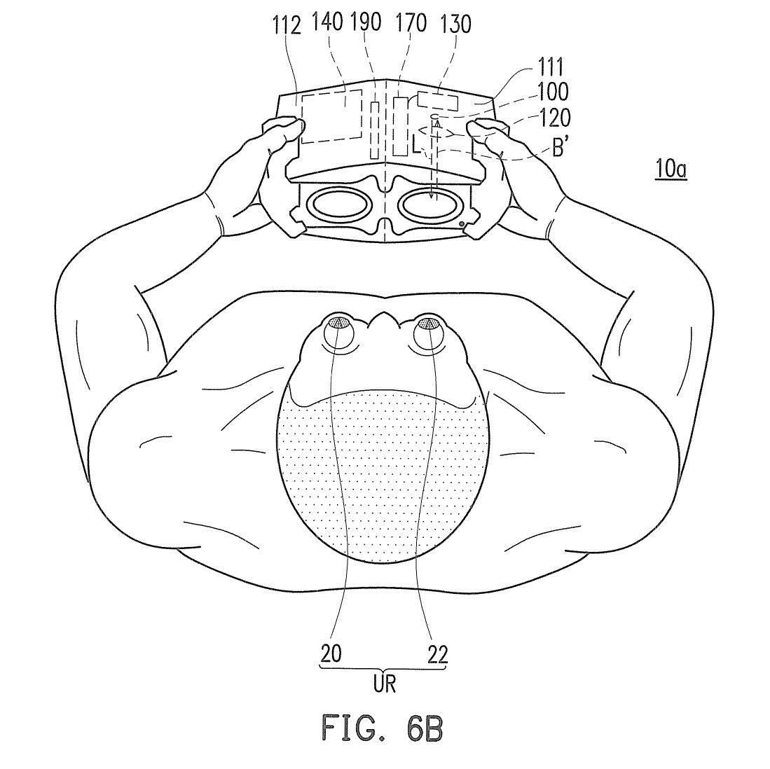

The image detecting apparatus 10a may further include an orientation sensor 190. As illustrated in FIG. 6A and FIG. 6B, when the image detecting apparatus 10a is flipped so that the positions of the first body 111a and the second body 112 are exchanged, the orientation sensor 190 may detect this exchange and may provide it to the control unit 170. With this, the control unit 170 may make a flip correction for the image detected by the image sensing device 130 according to this exchange, so that whether the user UR measures the first eyeball 20 of oneself (e.g., situation as illustrated in FIG. 6A) or measures the second eyeball 22 of oneself (e.g., situation illustrated in FIG. 6B), the user UR may observe from the display 140 the image substantially similar to the eye image detected by the image detecting apparatus 10a before being flipped, and thus enable the shooting of the eye image to be even more intuitive, so as to facilitate the user UR to shoot the eye image by oneself

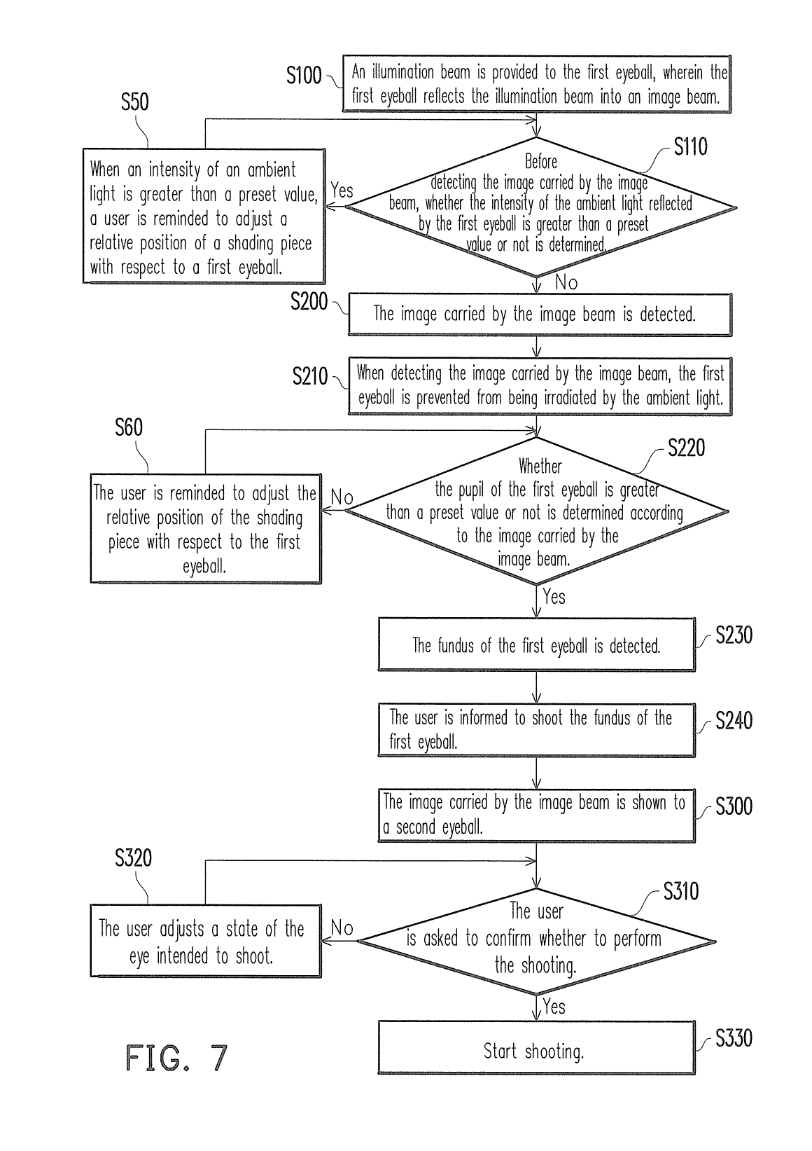

FIG. 7 is a flow chart diagram of an image detecting method according to an embodiment of the invention. Referring to FIG. 7 again, in this embodiment, the device structure may be referred to the image detecting apparatus in the embodiments illustrated in FIG. 1 and FIG. 3A, and is not repeated herein. The image detecting method includes providing an illumination beam L to the first eyeball 20, wherein the first eyeball 20 reflects the illumination beam L into the image beam B (step S100). In this embodiment, the illumination light source 100, for example, is configured to provide the illumination beam L. The image detecting method also includes detecting the image carried by the image beam B (step S200), and in this embodiment, the image sensing device 130, for example, is configured to sense the image formed by the image beam B. The image detecting method further includes showing the image carried by the image beam B to second eyeball 22 (step S300), wherein the first eyeball 20 and the second eyeball 22 belong to a same user UR. In this embodiment, the display 140, for example, is configured to show the image formed by the image beam B of the first eyeball 20 sensed by the image sensing device 130 to the second eyeball 22. As a result, the user UR not only detects the eye image of the first eyeball 20 thereof, but also assists in shooting or observing the eye image of the first eyeball 20 thereof with the second eyeball 22, thus enhancing an accuracy for shooting the eye image.

In addition, the image detecting method may further include preventing the first eyeball 20 from being irradiated by the ambient light when detecting the image carried by the image beam B (step S210). In this embodiment, the shading piece 160, for example, is configured to shield the light from the outside and to form a dark environment between the first eyeball 20 of the user and the image detecting apparatus 10, and thus reduce an influence of an ambient light to the first eyeball 20. Moreover, the image detecting method further includes determining an intensity of the ambient light reflected by the first eyeball 20 before detecting the image carried by the image beam B (step S110). In this embodiment, the control unit 170, for example, is configured to determine the intensity of the ambient light detected by the image sensing device 130. When the intensity of the ambient light is greater than a preset value, the user UR is reminded to adjust relative position of the first eyeball 20 with respect to the shading piece 160 (step S50) until the intensity of the ambient light is smaller than the preset value. In this embodiment, the control unit 170, for example, is configured to remind the user to adjust the shading piece 160 via the user interface 180, such that the shading piece 160 is closely fit on the first eyeball 20 until the intensity of the ambient light is smaller than the preset value. As a result, an influence to the eye image detection due to the ambient light may be reduced, so that the quality of the eye image may be enhanced to facilitate a diagnosis.

In this embodiment, the image detecting method may further include determining from the image carried by the image beam B whether the pupil P of the first eyeball 20 is greater than a preset value (step S220); and in this embodiment, the control unit 170, for example, is configured to determine a size of the pupil P sensed by the image sensing device 130. If not, then the user is reminded to adjust the relative position of the first eyeball 20 with respect to the shading piece 160 (step S60). In this embodiment, the control unit 170, for example, is configured to remind the user to adjust the positions of the eye and the image detecting apparatus 10 via the user interface 180. If yes, namely, the pupil P of the first eyeball 20 is greater than the preset value, then the fundus F of the first eyeball 20 is detected (step S230). In this embodiment, the control unit 170, for example, enters a standby shooting state. In addition, when the pupil P of the first eyeball 20 is greater than the preset value, then the user UR is informed to shoot the fundus F of the first eyeball 20 (step S240), and in this embodiment, for example, when the control unit 170 enters the standby shooting state, the control unit 170 informs the user to start shooting via the user interface 180. The means and details of informing the user may be referred to the embodiments illustrated in FIG. 1 and FIG. 2A to FIG. 2C, and thus are not repeated herein. The preset value for the size of the pupil P may be determined according to the actual needs, provided that the size is sufficient for the user to shoot the eye image for performing the diagnosis. With this, the user UR may obtain favorable eye image when shooting by oneself for providing to the physicians to perform the diagnosis.

In addition, after showing the image carried by the image beam B to a second eyeball 22 (step S300), the image detecting method may further include asking the user to confirm whether to perform the shooting (step S310). In this embodiment, the user interface 180, for example, is configured to inform the user to start shooting. If not, then the user UR is asked to adjust a state of the eye for intended shooting (step S320). For example, the user UR may observe the left eye image with the right eye, and thereby may adjust the left eye to a state for intended shooting, such as an angle or a position of the eye. If the user UR confirms to shoot, then the shooting starts (step S330). In this embodiment, the user, for example, may push the shooting button BT to shoot the eye image. With this, the user UR may still obtain favorable eye image by self-shooting without relying on instructional assistances from another person, and the convenience and efficiency for shooting the eye image may also be enhanced, such that the patient may shoot the eye images during any time at home, and use the remote medical facility system to transfer the eye images to the physicians for diagnosis and facilitating medical history tracking, thus saving the medical costs and improving the quality of medical care.

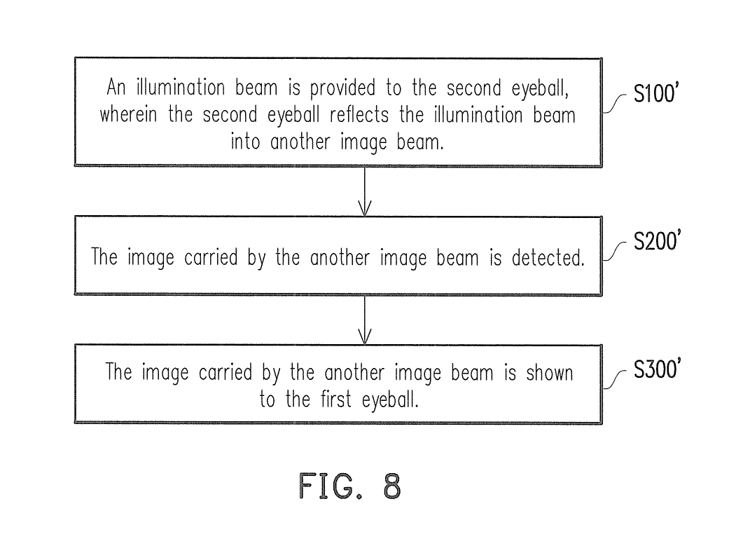

In more detail, as shown in a flow chart diagram illustrated in FIG. 8, the image detecting method of this embodiment may further include providing the illumination beam L to the second eyeball 22. As illustrated in FIG. 6B, the second eyeball 22 reflects the illumination beam L into another image beam B′ (step S100′). The image detecting method also includes detecting the image carried by another image beam B′ (step S200′). The image detecting method also includes showing the image carried by the image beam B′ to the first eyeball 20 (step 300′). Namely, as shown in FIG. 6A and FIG. 6B, the image detecting apparatus 10a is flipped in angle to exchange the positions of the first body 111 and the second body 112, i.e. to exchange the measured eye with the observing eye; and therefore the user UR may respectively measure the eye images of the first eyeball 20 and the second eyeball 22 thereof, so as to provide a more complete image information to the physicians for diagnosis.

In summary, in the embodiments of the invention, the user may align the pupil and attain the favorable image shooting condition via visual or auditory prompts from the user interface in the image detecting apparatus or the eye images shown by the display, and thus capable of shooting the images of the fundus by oneself so as to facilitate the physicians to perform diagnosis and tracking. Moreover, the image detecting apparatus may also transform, causing the display and the imaging lens to face towards the opposite directions, and may enable an operator to observe the eye image of a subject shown by the display, and thereby may also enable the image detecting apparatus to be used by another person. In addition, the eye of the user may be surrounded by the shading piece, and therefore may prevent the ambient light from influencing the measurement of the eye images. Moreover, when the illumination light source is an invisible light source, such as infrared, in collocation with the shading piece, a dark environment is formed between the eye to be measured and the imaging lens, so that the pupil of the eye to be measured is naturally dilated to facilitate shooting the eye images; and therefore, the side effects brought by using the mydriatic agent may be avoided, thereby may reducing the burdens on the user due to a long-term tracking of the eye images.

It will be apparent to those skilled in the art that various modifications and variations can be made to the structure of the invention without departing from the scope or spirit of the invention. In view of the foregoing, it is intended that the invention cover modifications and variations of this invention provided they fall within the scope of the following claims and their equivalents.

What is claimed is:

1. An image detecting apparatus for detecting a first eyeball, the image detecting apparatus comprising:
an illumination light source emitting an illumination beam, the illumination beam irradiating the first eyeball, the first eyeball reflecting the illumination beam into an image beam;
an imaging lens disposed on a transmission path of the image beam;
an image sensing device disposed on the transmission path of the image beam, wherein the imaging lens is disposed between the first eyeball and the image sensing device;
a display showing an image formed by the image beam that are sensed by the image sensing device; and
a viewfinder disposed in front of the display, such that a second eyeball observes the display via the viewfinder.

2. The image detecting apparatus as recited in claim 1, wherein the illumination beam and the image beam are invisible beams.

3. The image detecting apparatus as recited in claim 2 further comprising a shading piece disposed on the imaging lens and surrounding a space between the imaging lens and the first eyeball.

4. The image detecting apparatus as recited in claim 3 further comprising:
a control unit electrically connected to the image sensing device; and
a user interface electrically connected to the control unit, wherein when the control unit determines that an intensity of an ambient light detected by the image sensing device is greater than a preset value, the control unit reminds the user to adjust the shading piece via the user interface, such that the shading piece is closely fit on the first eyeball.

5. The image detecting apparatus as recited in claim 1 further comprising a control unit electrically connected to the imaging lens and the image sensing device, wherein the control unit commands the imaging lens to focus on a pupil of the first eyeball, and when the control unit determines an image size of the pupil sensed by the image sensing device is larger than a preset value, the control unit enters a standby shooting state.

6. The image detecting apparatus as recited in claim 5, wherein when the control unit enters the standby shooting state, the control unit commands the imaging lens to focus on a fundus of the first eyeball.

7. The image detecting apparatus as recited in claim 5 further comprising a user interface, wherein when the control unit enters the standby shooting state, the control unit informs the user to start shooting via the user interface.

8. The image detecting apparatus as recited in claim 1 further comprising:
a first body carrying the illumination light source, the imaging lens and the image sensing device; and
a second body carrying the display and the viewfinder, wherein the second body is adapted to be detachably bound to or rotatably bound to a first position or a second position on the first body, when the second body is bound to the first position on the first body, the imaging lens and the viewfinder face towards a same direction, and when the second body is bound to the second position on the first body, the imaging lens and the viewfinder respectively face towards opposite directions.

9. The image detecting apparatus as recited in claim 8, wherein when the second body is bound to the first position on the first body, the imaging lens and the viewfinder respectively face towards the first eyeball and the second eyeball, and the first eyeball and the second eyeball both belong to a same user.

10. The image detecting apparatus as recited in claim 8, wherein when the second body is bound to the second position on the first body, the imaging lens and the vie finder respectively face towards the first eyeball and the second eyeball, and the first eyeball and the second eyeball respectively belong to a subject and an operator.

11. The image detecting apparatus as recited in claim 8, wherein when the second body is bound to the second position on the first body, the display moves from within the second body to an external part of the second body, the imaging lens and the display respectively face towards the first eyeball and the second eyeball, and the first eyeball and the second eyeball respectively belong to a subject and an operator.

12. The image detecting apparatus as recited in claim 8, wherein the first body has a first handle and the second body has a second handle, and when the second body is bound to the first position on the first body, the first handle and the second handle are respectively located at two opposite sides of the image detecting apparatus, so as to be respectively gripped by two hands of a user.

13. The image detecting apparatus as recited in claim 8, wherein at least one of the first body and the second body has a foot stand fixing hole for fixing a foot stand configured to support the image detecting apparatus.

14. The image detecting apparatus as recited in claim 1 further comprising a control unit electrically connected to the image sensing device and the display, wherein when the control unit determines that a pupil of the first eyeball detect by the image sensing device is deviated from a central region of the image sensing device, the control unit commands the display to show a prompting sign to inform the user on how to move a relative position of the imaging lens with respect to the first eyeball.

15. An image detecting method comprising:
providing an illumination beam to a first eyeball, wherein the first eyeball reflects the illumination beam into an image beam;
detecting an image carried by the image beam; and
showing the image carried by the image beam to a second eyeball, wherein the first eyeball and the second eyeball belong to a same user.

16. The image detecting method as recited in claim 15 further comprising:
determining an intensity of an ambient light reflected by the first eyeball before detecting the image carried by the image beam, wherein when the intensity of the ambient light is greater than a preset value, the user is reminded to adjust a relative position of a shading piece with respect to the first eyeball until the intensity of the ambient light is less than the preset value.

17. The image detecting method as recited in claim 15 further comprising determining whether a pupil of the first eyeball is greater than a preset value or not according to the image carried by the image beam, wherein if not, then the user is reminded to adjust the relative position of a shading piece with respect to the first eyeball.

18. The image detecting method as recited in claim 17, wherein if the pupil of the first eyeball is greater than the preset value, then a fundus of the first eyeball is detected.

19. The image detecting method as recited in claim 17, wherein if the pupil of the first eyeball is greater than the preset value, then the user is informed to start shooting a fundus of the first eyeball.

20. The image detecting method as recited in claim 15 further comprising:
providing the illumination beam to the second eyeball, wherein the second eyeball reflects the illumination beam into another image beam;
detecting an image carried by the another image beam; and
showing the image carried by the another image beam to the first eyeball.
